(19) United States
(12) Patent Application Publication
(10) Pub. No.: US 2025/0360332 A1
(43) Pub. Date: Nov. 27, 2025

(54) PHOTOTHERAPY APPARATUS FOR TREATING ALZHEIMER'S DISEASE

(71) Applicant: DANYANG HUICHUANG MEDICAL EQUIPMENT CO., LTD., Jiangsu (CN)

(72) Inventor: Daifa WANG, Jiangsu (CN)

(21) Appl. No.: 18/998,364

(22) PCT Filed: Jul. 26, 2023
(86) PCT No.: PCT/CN2023/109292

§ 371 Date: Jan. 24, 2025

(30) Foreign Application Priority Data
Jul. 26, 2022 (CN) 202210883901.4
Jul. 26, 2022 (CN) 202210886242.X
Jul. 26, 2022 (CN) 202210886248.7
Jul. 26, 2022 (CN) 202210886262.7

(51) Int. Cl. A61N 5/06; A61N 5/00

(52) U.S. Cl. CPC A61N 5/0622; A61N 2005/007; A61N 2005/0627; A61N 2005/0632; A61N 2005/0647; A61N 2005/0652; A61N 2005/0658

(57) ABSTRACT

The present application relates to a phototherapy apparatus for treating Alzheimer's disease. The apparatus comprises: a hood body that loosely accommodates the patient's head; an array of near-infrared irradiation units arranged in the hood body, which is constructed to emit near-infrared light with an average irradiance greater than 40 mW/cm² towards the head; and a cooling mechanism configured to introduce cooled gas and send it into the cover, blowing it towards the patient's head through a cooled gas delivering pathway between the hood body and the patient's head to dissipate heat from the patient's head. This device can effectively treat patients with Alzheimer's disease, adapt to the special psychological and physiological needs of patients with various disease courses, improve patients' treatment compliance, and ensure good treatment effect for Alzheimer's disease.

### TECHNICAL FIELD

The present disclosure relates to a technical field of medical equipment, especially relates to a phototherapy apparatus for treating Alzheimer's disease.

### BACKGROUND

Epidemiological survey shows that the incidence rate of human cerebral functional related diseases is on the rise in modern society, among which Alzheimer's disease (AD) is one of the most common cerebral functional related diseases.

AD is more common in elderly people over 65 years old, with slow or hidden onset. It deteriorates over time, mainly manifested as cognitive decline, language dysfunction, emotional instability, mental symptoms and behavioral disorders, gradual decline in daily living ability, and ultimately loss of physical function and leading to death. At present, the causes of AD are unclear. With the development of global population aging, the number of AD patients continues to increase, bringing a heavy burden to families and society.

There is currently no effective drug therapy for AD in clinical practice. Over the years, a large number of researches have been carried out at home and abroad on the use of physical electromagnetic stimulation to treat AD, such as transcranial direct current stimulation, transcranial magnetic therapy, etc., but these electromagnetic treatments can only reach the cerebral cortex, not the deep encephalic region. In recent years, cutting-edge research on the use of near-infrared light to treat Alzheimer's disease has also been conducted both at home and abroad. However, although there are some research results, in vivo experiments are mostly conducted on mice, and there are few clinical results with human subjects.

The average irradiance of near-infrared light of phototherapy apparatuses for treating Alzheimer's disease in the prior art is relatively low, and the energy deposition of near-infrared light on the brain tissue after penetrating the skull is less, making it difficult to achieve good phototherapy effect. The near-infrared treatment modules are set separately, making it impossible for near-infrared light to reach the whole brain, resulting in poor phototherapy effect and potential safety issues caused by the leakage of near-infrared light.

Furthermore, the hood body of existing phototherapy apparatuses that use near-infrared light to treat AD is usually adapted to (or mounted to) the shape of the patient's head. In addition to decreased memory ability, AD patients may also exhibit emotional agitation, anxiety, lack of security, often suspicious, and easily angered. For example, AD patients may have their head clamped or required to keep their head still during treatment, which can lead to strong resistance, poor acceptance and compliance to phototherapy of the patients, thus affecting the effectiveness of phototherapy.

### SUMMARY

The present application is provided to address the aforementioned issues in prior art. A phototherapy apparatus for treating Alzheimer's disease is needed, which can effectively treat patients with Alzheimer's disease, adapt to the special psychological and physiological needs of patients with Alzheimer's disease of various disease courses, improve patients' treatment compliance, and ensure good treatment effect for Alzheimer's disease.

According to the first aspect of the present application, a phototherapy apparatus for treating Alzheimer's disease is provided. The phototherapy apparatus includes a hood body that loosely accommodates the patient's head, so as to enable the head to rotate within a predetermined angle range and move in the up-down direction within a predetermined distance range during the treatment. The phototherapy apparatus includes an array of near-infrared irradiation units arranged in the hood body, which is constructed to emit near-infrared light with an average irradiance greater than 40 mW/cm² towards the patient's head. The phototherapy apparatus also includes a cooling mechanism configured to introduce cooled gas and send it into the hood body, blowing it towards the patient's head through a cooled gas delivering pathway between the hood body and the patient's head to dissipate the heat of the patient's head.

With the phototherapy apparatus for treating Alzheimer's disease according to various embodiments of the present application, it can effectively treat patients with Alzheimer's disease, adapt to patients with Alzheimer's disease of various disease courses, and enable patients with Alzheimer's disease to be in a relatively comfortable environment during the treatment, especially suitable for patients with Alzheimer's disease who are intolerant to temperature rise meanwhile requiring higher irradiation power and other physiological needs, improving patients' treatment compliance, and being able to meet special psychological needs such as emotional agitation, anxiety, psychological disorder in confined and crowded space, ensuring good treatment effect for Alzheimer's disease.

### BRIEF DESCRIPTION OF THE DRAWINGS

In figures that are not necessarily drawn to scale, the same reference numerals may describe similar components in different figures. The figures generally show various embodiments by way of example rather than limitation, and are used together with the description and the claims to describe the embodiments of the present disclosure. As proper, the same reference sign may be used throughout the drawings to denote the same or similar part. Such embodiments are illustrative, and are not intended to be exhaustive or exclusive embodiments of the present device or method.

FIG. 1(a) shows a structural schematic diagram of a phototherapy apparatus for treating Alzheimer's disease according to the embodiment of the present application;

FIG. 1(b) shows a structural schematic diagram of the array of near-infrared irradiation units in the headgear of a phototherapy apparatus for treating Alzheimer's disease;

FIG. 2 shows a schematic diagram of the distribution of various brain regions in the whole brain of the patient according to the embodiment of the present application;

FIG. 3(a) shows a comparative diagram of energy deposition in the dorsal lateral prefrontal cortex (dlPFC) of individuals of different ages when they are irradiated with near-infrared light of various wavelengths according to the embodiment of the present application;

FIG. 3(b) shows a comparative diagram of energy deposition in the ventromedial prefrontal cortex (vmPFC) of individuals of different ages when they are irradiated with near-infrared light of various wavelengths according to the embodiment of the present application;

FIG. 4 shows the absorption curves diagram of near-infrared light of different wavelengths in water, deoxyhemoglobin, and oxyhemoglobin according to the embodiment of the present application;

FIG. 5 shows a schematic diagram of the headgear of a phototherapy apparatus for treating Alzheimer's disease according to the embodiment of the present application in the wearing state;

FIG. 6 shows a perspective view of the headgear of the phototherapy apparatus for treating Alzheimer's disease according to the embodiment of the present application;

FIG. 7 shows the arrangement diagram of near-infrared LEDs on a lamp panel as an example of a near-infrared irradiation unit in a phototherapy apparatus for treating Alzheimer's disease;

FIG. 8 shows a overall structural schematic diagram of the phototherapy apparatus for treating Alzheimer's disease according to the embodiment of the present application;

FIG. 9 shows a comparison chart of in vivo experimental data of the water maze experiment between AD mice receiving phototherapy and comparative group of AD mice after performing phototherapy on AD mice using the illumination parameter of the phototherapy apparatus according to the embodiment of the present application on 5-month-old AD mice;

FIG. 10 shows the pathological section diagram of the cerebral cortex area and hippocampus CA1 area of the AD mice receiving phototherapy and the comparative group of AD mice after performing phototherapy on the 5-month-old AD mice with the illumination parameter of the phototherapy apparatus according to the embodiment of the application, in which the distribution of β-amyloid protein (Aβ) (which is shown as particles in FIG. 10) is shown; and

FIG. 11 shows a comparison chart of the Alzheimer's Disease Assessment Scale Cognitive Scale (ADAS-cog) scores between AD patients receiving phototherapy and comparative group of AD patients after performing phototherapy on AD patients using the phototherapy apparatus according to the embodiment of the present application.

### DETAILED DESCRIPTION

In order to enable those skilled in the art to better understand the technical solutions of the present application, the present application will be described in detail below in conjunction with the accompanying drawings and specific embodiments. Embodiments of the present application will be described in further detail below in conjunction with the accompanying drawings and specific embodiments, but this is not intended to limit the present application. For the various steps described herein, if there is no need for a contextual relationship between each other, the order described herein as an example should not be considered as a limitation, and those skilled in the art will know that the order can be adjusted, as long as the logic between them is not disrupted, making the entire process impossible to implement.

“First”, “second” and similar words used in the present application do not indicate any sequence, quantity or importance, but are only used for distinction. Words like “including” or “comprising” mean that the elements preceding the word cover the elements listed after the word, and do not exclude the possibility of also covering other elements. The term “head” used in the present application refers to the organs above the neck (cervical spine) of the human body, including the brain and extracerebral tissues such as skull, skin and hair. The term “brain” used in the present application refers to an organ left after removal of the extracerebral tissues, etc., and is mainly intended to refer to the cerebrum, but is not limited thereto, and may also include the cerebrum, cerebellum, and brainstem. The term “whole brain” as used in the present application is intended to be distinguished from discrete encephalic regions such as frontal lobe and temporal lobe, but is not limited to exhaustive regions of the “brain”. “Whole brain” at least includes the frontal lobe, temporal lobe, parietal lobe, and occipital lobe, and in some cases may (but does not have to) further include hippocampus, amygdala, etc., and in others cases may (but does not have to) further include cerebellum, brainstem, and the like.

The applicant's research team has conducted in-depth research on the treatment of AD with near-infrared light and phototherapy apparatus, and conducted a large number of simulation experiments and clinical experiments. The applicant not only demonstrates the theoretical and practical feasibility of phototherapy apparatus for treating AD, but also focuses on and conducts in-depth research on the special psychological and physiological needs of patients with AD in clinical experiments targeting patients with AD of various disease courses. The present application proposes a phototherapy apparatus for treating Alzheimer's disease, which can not only significantly improve the patient's treatment compliance, but also ensure a good treatment effect on AD.

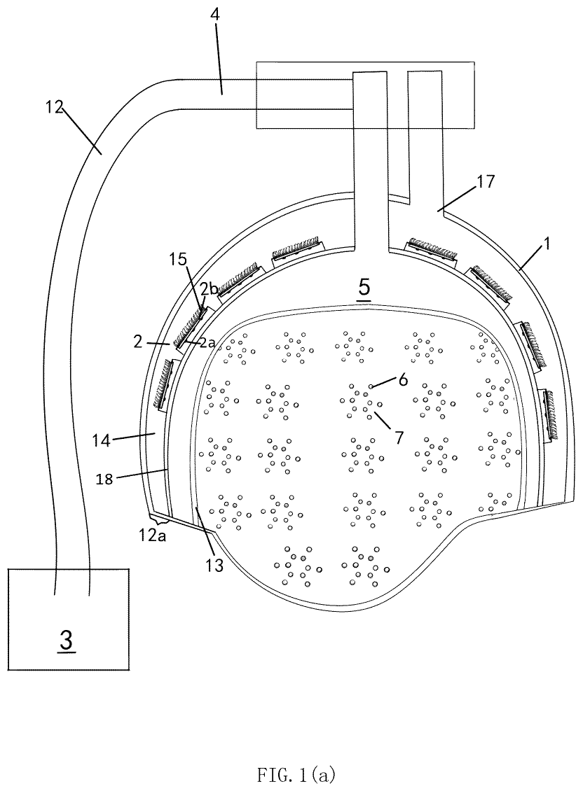

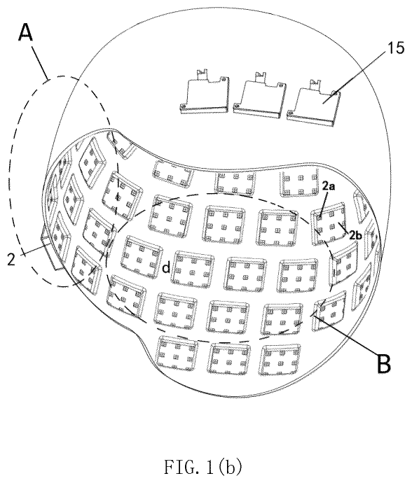

FIG. 1(a) shows a schematic structural view of a phototherapy apparatus for treating Alzheimer's disease according to an embodiment of the present application. As shown in FIG. 1(a), the phototherapy apparatus includes a hood body 1, which loosely accommodates the patient's head so that the head can rotate within a preset angle range and can move in the up-down direction within the preset distance range during the treatment. Different from adapting to the shape of the patient's head, a gap of a few centimeters (a gap at centimeter-level) is reserved between the hood body 1 and the patient's head. Preferably, the gap is 1-2 cm, so that the patient can move his/her own head within the predetermined angle range and within the predetermined distance range according to his own wishes. This open design of hood body 1 has no sense of restraint on the patient's head, and is particularly friendly for elderly people who are emotionally agitated, anxious, resistant, or even afraid of confined or crowded spaces, which can significantly improve the treatment compliance of patients with AD.

In particular, the phototherapy apparatus may be used to treat patients with AD and with psychological disorder to confined or crowded space. This psychological disorder to confined or crowded space can be caused by the patient's own age or AD the patient suffers. This design of the hood body 1 can also be widely applied to the behavioral characteristics of patients in different courses of AD. For example, for early AD patients, the judgment ability is reduced and they are often suspicious and can be provoked. The wearing of this kind of hood body 1 with sufficient degree of freedom and openness is easy to be accepted by patients and is not easy to bring about irritation, so that patients can cooperate with the continuation of phototherapy. For another example, for AD patients in the middle stage, their emotions fluctuate violently, they are irritable and restless, and they often walk around frequently. Some patients's head may vibrate frequently with small amplitudes without consciousness. This open hood body 1 allows the performing of the frequent and unconscious vibration of their heads with small amplitude without causing the hood body 1 itself to vibrate together with the head. Therefore, it is unnecessary to forcibly stop this small-amplitude vibration, which increases the comfort of the patient and reduces the workload of the medical staff. At the same time, it can also prevent the vibration of the patient's head from being delivered to the hood body 1, which will affect the phototherapy effect negatively. Therefore, the phototherapy apparatus can be used to treat patients with Alzheimer's disease who are restless and anxious.

The hood body 1 with open and loose design can make AD patients during various disease courses more willing to accept treatment. A single irradiation fraction can last for a longer time, such as 20 minutes, 30 minutes, or even longer each time, so as to further enhance the treatment effect.

The phototherapy apparatus further includes an array of near-infrared irradiation units 2 arranged in the hood body 1. The array of near-infrared irradiation unit 2 is arranged corresponding to various brain regions of the head. As an example, as shown in FIG. 1(b), each near-infrared irradiation unit 2 includes a plurality of near-infrared light-emitting diodes 2a. In some embodiments, each near-infrared irradiation unit 2 may include a single near-infrared light emitting diode 2a. The array of near-infrared irradiation units 2 is constructed to emit near-infrared light with an average irradiance greater than 40 mW/cm² to the patient's head. It can be understood that the term “average irradiance” used in the present application means the magnitude of energy of near-infrared light irradiation on a unit area per unit time.

The inventor found through research that for elderly patients with AD such as those over 65 years old, higher irradiance needs to be provided to ensure that enough light energy enters the brain to effectively treat AD. In the phototherapy apparatus of the embodiment of the present application, the array of near-infrared irradiation units 2 is constructed to emit near-infrared light with an irradiance greater than 40 mW/cm² towards the patient's head, so that enough light energy enters the brain. Furthermore, in the phototherapy apparatus of the embodiment of the present application, the hood body 1 does not adopt a fitting design, but adopts a loose design in which the head can move freely in the accommodating space, and the gap between the hood body 1 and the head will lead to the scattering of near-infrared light, and the scattering and superposition of near-infrared light make each brain region have a higher irradiance, which further meets the demand for increasing irradiance, and then meets the higher irradiance requirements of the whole brain.

The inventor found through simulation and clinical experiments that, for the phototherapy apparatus with loose disigned hood body 1 of this application, an average irradiance greater than 40 mW/cm² is adopted, which can also ensure that sufficient light energy enters the brain tissue for elderly people with AD, ensuring good treatment effect. Specifically, the average irradiance used can be 40 mW/cm²-150 mW/cm², such as 60 mW/cm², 70 mW/cm², 80 mW/cm², 90 mW/cm², 100 mW/cm², 120 mW/cm² and so on. For some specific encephalic regions, such as frontal lobe and temporal lobe, the average irradiance of near-infrared light can be above 50 mW/cm² to 250 mW/cm², preferably above 70 mW/cm².

When adopting an average irradiance greater than 40 mW/cm², the conventional fan-type cooling mechanisms are not suitable and does not work well. Even if the air volume is increased, AD patients still feel discomfort with the thalposis near the scalp, or even an unbearable hot sensation, thus they cannot withstand continuous treatment, and the excessive air volume will also cause discomfort to the patient's head. However, for some types of radiation therapy, such as radiation therapy of the whole encephalic regions (or “whole brain”), or for some special focused brain regions, such as the frontal lobe and temporal lobe, the required average irradiance may be higher, such as reaching 70 mW/cm² or more, even 150 mW/cm². By irradiating cortical cells with near-infrared light in vitro, it was demonstrated that an array of near-infrared irradiation units 2 can be constructed to emit near-infrared light with an average irradiance less than 250 mW/cm² to the patient's head, and the average irradiance in this range can avoid the risk of thermal damage and avoid inhibition and mitochondrial damage.

In some embodiments, the required average irradiance of the employed near-infrared light can be determined and adjusted according to the characteristics of the patient. For example, the average irradiance is determined according to the light transmittance of the extracerebral tissue of the patient, so that the average irradiance for the patient whose extra-brain tissue has low light transmittance is higher than the average irradiance for the patient whose extra-brain tissue has high light transmittance.

Specifically, AD patients have lower sensitivity to temperature and pain, that is, AD patients may experience greater pain and potentially greater tissue or organ damage before damage is detected and reported, and, in AD patients, the elderly over 65 years old account for a large proportion, and the elderly are more likely to be afraid of the cold than the young. Therefore, in the case of using a higher average irradiance or even higher total power to treat AD patients, it is especially necessary to fully consider the sensitivity of AD patients to temperature and pain, and provide them with a more comfortable environment that does not cause pain and even thermal damage, which helps to prolong the treatment time, thus enabling better treatment result.

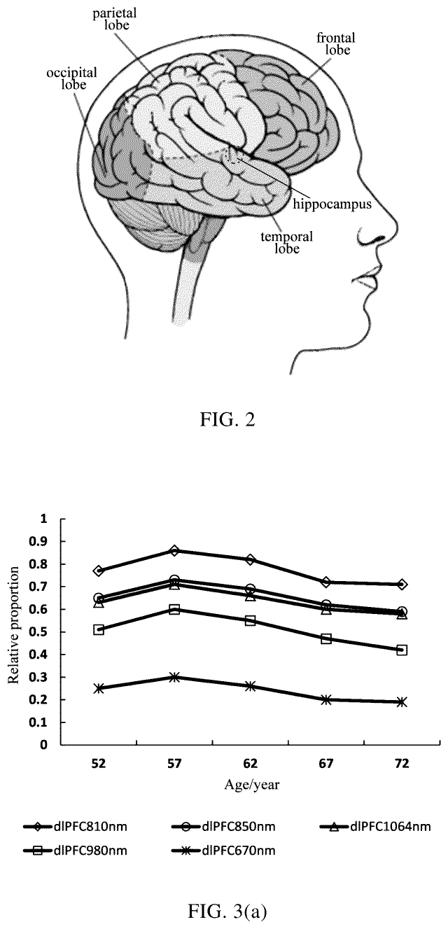

FIG. 2 shows a schematic diagram of the distribution of various brain regions of the cerebral cortex of the whole brain of a patient according to an embodiment of the present application. As shown in FIG. 2, the various brain regions of the cerebral cortex mainly include the frontal lobe, the temporal lobe, the parietal lobe, the occipital lobe, and the cerebellum, etc.

The inventor found that in the treatment of AD, the array of near-infrared irradiation units 2 can emit near-infrared light to all brain regions of the patient's head together, especially to the frontal lobe, temporal lobe and hippocampus at least, which can achieve better treatment effect than only irradiating some brain regions. Specifically, as shown in FIG. 2, the hippocampus is located between the thalamus and the medial temporal lobe. And the hippocampus is part of the limbic system and plays a role in short-term memory, long-term memory and spatial positioning. Hippocampus atrophy is closely related to Alzheimer's disease. With the development of AD, the lesions further spread to the frontal lobe and the temporal lobe, and the brain slowly shrinks, leading to further loss of memory and loss of self-help ability. The functions of the temporal lobe mainly include auditory perception, language reception, visual memory, declarative (true) memory, and emotional control, etc. Specifically, patients with right temporal lobe lesions often lose their understanding of non-verbal auditory stimuli (such as music, etc.), while left temporal lobe lesions can affect patients' perception, memory, and organization of language. The frontal lobe is the physiological basis of the most complex psychological activities of human beings. It is responsible for planning, regulating and controlling human psychological activities, and plays an important role in the advanced and purposeful behavior of human beings. It is closely related to advanced cognitive functions such as attention, memory, and problem-solving, as well as personality development.

By allowing the hood body 1 to cover the whole brain, the array of near-infrared irradiation units 2 can emit near-infrared light to all the brain regions of the patient's head together, including at least the frontal lobe, the temporal lobe and the hippocampus, so as to conduct comprehensive and thoughtful phototherapy on the cortical regions involved in the lesion, so as to achieve better treatment effect (which will be confirmed by clinical experiments and clinical data herein after). In some embodiments, the array of near-infrared irradiation unit 2 is constructed to emit near-infrared light to the frontal lobe and the temporal lobe with an average irradiance higher than that for other brain regions, thereby enhancing the treatment effect on the focused frontal lobe and temporal lobe.

In some embodiments, the array of near-infrared irradiation units 2 is constructed to emit near-infrared light to nodes of the brain network, which may at least include at least one of the medial prefrontal cortex, medial temporal lobe, cingulate cortex, and precuneus and inferior parietal lobe. Studies have shown that there is a certain correlation between the functional connections between the nodes of the brain network in the medial prefrontal cortex, medial temporal lobe, cingulate cortex, precuneus, and inferior parietal lobe and the development process of AD. For example, brain network connections are constructed for AD patients, and it was found that, compared with normal people, the functional connection strength between the hippocampus located in the medial temporal lobe and the medial prefrontal cortex, precuneus and other nodes of the brain network was significantly weakened. Even the functional connection strength results of some AD patients' brain networks show that the hippocampus has lost connectivity with certain nodes of the brain network. The array of near-infrared irradiation unit 2 is constructed to emit near-infrared light to the above-mentioned nodes of the brain network, which can enhance the brain functional connectivity between these nodes of the brain network, improve the collaborative work and information transmission capabilities of brain regions, and improve memory ability and cognitive functions. Specifically, a brain network can be constructed based on cerebral functional imaging and/or brain structural imaging of the patient's head, and various nodes of the brain network can be determined. For example, a brain network can be established through MRI images, which can include multiple nodes with brain regions as nodes, various nodes correspond to different brain regions. Alternatively, multiple nodes can be set on one brain region, with functional connections between node in pairs. The functional connection strength between the node in pairs can be used to characterize the collaborative work and information transmission capabilities between brain regions, etc.

In a specific embodiment, the nodes of the brain network at least include the hippocampus located in the medial temporal lobe, and the array of near-infrared irradiation units 2 is constructed to emit near-infrared light to the hippocampus and to the nodes of the brain network, the functional connection strength of which with the hippocampus is weaker than a predetermined level. In this way, irradiation is applied to nodes in the brain network that have imbalanced functional connections (i.e., functional connections are weaker than predetermined levels, such as functional connections are weaker than normal level or losing functional connections) by means of targeting at and focusing locally on these nodes. For example, using near-infrared light with an average irradiance greater than 70 mW/cm² to irradiate can not only enhance the functional connections strength between the hippocampus and other nodes of the brain network, but also even help to restore functional connections between the hippocampus and other nodes of the brain network, achieving good treatment effect on AD. Only emitting near-infrared light to these brain network node areas can reduce heat production, reduce requirements for cooling mechanisms, and make it easier to keep the patient's head in a more comfortable environment, improving comfort level.

In some embodiments, the array of near-infrared irradiation unit 2 is constructed to emit near-infrared light together to these nodes of the brain network including the medial prefrontal cortex, hippocampus located in the medial temporal lobe, anterior cingulate cortex, precuneus, and inferior parietal lobe. This can enhance the functional connectivity between various nodes in the brain network and achieve better treatment effect on AD.

It can be understood that when the array of near-infrared irradiation units 2 is used to irradiate the nodes of the brain network locally in a targeted manner, for a node pair with imbalanced functional connection, one node of the node pair can be irradiated, or the node pair (that is, two nodes) can be irradiated together. Especially in the case that one node of the node pair is located deep in the brain and is difficult to be irradiated, such as the hippocampus located deep in the brain, the other node of the node pair that is easier to be irradiated is irradiated, which can also indirectly act on the other node located in the deep part of the brain to achieve a certain phototherapy effect. The present application does not specifically limit the irradiating manner of nodes of the brain network.

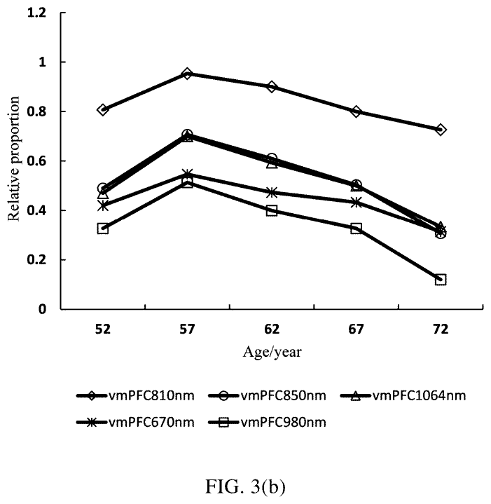

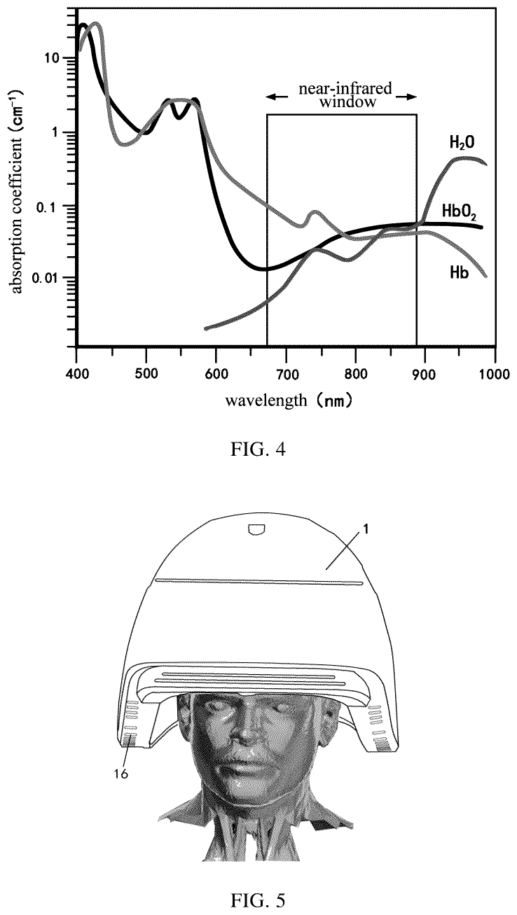

In some embodiments, the array of near-infrared irradiation unit 2 can be constructed to emit near-infrared light with a certain range of central wavelength towards the patient's head, so that the average energy deposition of near-infrared light and absorption coefficients of deoxyhemoglobin (Hb) and oxyhemoglobin (HbO₂) are good. Preferably, the array of the near-infrared irradiation units 2 can be configured to emit near-infrared light with a central wavelength of 800-820 nm towards the patient's head, which will be explained in detail in conjunction with FIG. 3(a), FIG. 3(b), and FIG. 4 below. Preferably, the array of the near-infrared irradiation units 2 is configured to emit a single wavelength near-infrared light with a central wavelength of 810 nm towards the patient's head.

The phototherapy apparatus according to the embodiment of the present application further includes a cooling mechanism 4 to sufficiently dissipate heat from the patient's head so as to well solve the above problems. Specifically, the cooling mechanism 4 is configured to introduce cooled gas and send it into the hood body 1, and blow it to the patient's head through the cooled gas delivering pathway between the hood body 1 and the patient's head, so as to dissipate heat from the patient's head. Wherein, the temperature of the cooled gas introduced by the cooling mechanism 4 is lower than the surface temperature of the patient's head.

In some embodiments, as shown in FIG. 1(a), the cooling mechanism 4 may include a refrigerator 3 or be connected to the refrigerator 3, and the hood body 1 is provided with a cooled gas delivering inner cavity 5 to receive the cooled gas generated by the refrigerator 3. The cooled gas delivering pathway 7 is directly formed between the inner wall of the cooled gas delivering inner cavity 5 and the patient's head. The cooled gas delivering inner cavity 5 can be set in various ways, for example, the cooled gas delivering inner cavity 5 and the near-infrared irradiation unit holding chamber (not shown) and the like can be discretely set in the internal open space of the hood body 1. The cooled gas delivering inner cavity 5 can be integrated with other chambers in the hood body 1, arranged in layers, or arranged in a staggered manner independently, which is not specifically limited. In some embodiments, a refrigeration device other than the refrigerator can be adapted.

In some embodiments, the cooled gas is blown towards the patient's head through the cooled gas delivering pathway 7 with a wind speed of 0.5 m/s to 3.5 m/s, which is comfortable for the patient and can ensure the heat dissipating effect. Preferably, the wind speed is 1 m/s-2.5 m/s. With such a cooling mechanism 4, when the array of near-infrared irradiation units 2 together emits the near-infrared light to the patient's head (for example, the whole encephalic regions), the total power of the near-infrared light is greater than 3 W, even for some special phototherapy schedules that have higher requirements for total power, the total power can be up to 10 W or more. Under the condition of such average irradiance and total power, the cooling mechanism 4 can still sufficiently dissipate heat from the patient's head, so that the temperature near the patient's scalp is 23° C. to 43° C. Preferably, the temperature near the patient's scalp is 25° C. to 40° C., which is close to body temperature. The human body will be more comfortable in this temperature environment, even the elderly who are less sensitive to temperature and pain and are afraid of cold will feel more comfortable and will not cause heat damage, allowing them to continue receiving treatment. Through such a cooling mechanism 4 combined with the previously described loosely designed hood body 1, patients with various disease courses are more willing to receive continuous irradiation treatment, and a single fraction of irradiation can last for a longer time (the longer the time is, the higher the heat production near the scalp is), thereby further enhancing the treatment effect.

As an example, the cooled gas delivering pathway 7 can be formed by the ventilation hole 6 inside the cooled gas delivering inner cavity 5 and the gap between the hood body 1 and the patient's head, as shown in FIG. 1(a), but this is merely an example, the cooled gas delivery tube may also be led out from the cooled gas delivering inner cavity 5 to deliver the cooled gas toward the head of the patient, which will not be described in detail here. By using the structure of cooling mechanism 4, hood body 1 covers the head in all directions, which avoids light leakage, reduces the safety risk caused by infrared light leakage, and realizes the transmission of sufficient light energy to the whole brain to ensure the treatment effect, and at the same time still ensures a good and comfortable cooling effect.

The above structural features are described in detail in the Chinese application as priority (with the application number 202210886242X), the relevant contents of which are incorporated herein as examples.

FIG. 5 shows a schematic diagram of a headgear of the phototherapy apparatus for treating Alzheimer's disease according to an embodiment of the present application in a wearing state. As shown in FIG. 5, the hood body 1 has a fixed structure and size, and accommodates the patient's head in a loose manner, so that the lateral movable interval of the patient's head is 1-2 cm during treatment. In some embodiments, the hood body 1 is constructed to cover at least the whole brain. Specifically, the structure of the hood body 1 leaves a certain margin, so that when the patient rotates within the predetermined angle range or moves in the up-down direction within the predetermined distance range, the hood body 1 can still cover the whole brain, so that various brain regions of the whole brain can be irradiated using the array of near-infrared irradiation units 2 (see FIG. 1(a) and FIG. 1(b)) if necessary. Further, with the loose and open design of the hood body 1, a fixed structure and size may be adopted for patients with individual differences in the shape and size of the head to a certain extent, and it is not necessary to customize the hood body 1 that strictly matches the individual shape and size of the head for the individual patient, so that the hood body 1 can be manufactured in a standardized manner, the manufacturing cost is lower. Thus, the phototherapy apparatus can adapt to a wider range of patient groups, thereby reducing the procurement and maintenance costs of the phototherapy apparatus in places such as hospitals, communities, and family facilities. Specifically, the so-called fixed structure and size mean that the hood body 1 may not be provided with movable components, and may even be integrally molded, thereby increasing the service life of the hood body 1 and simplifying the structure of the hood body 1.

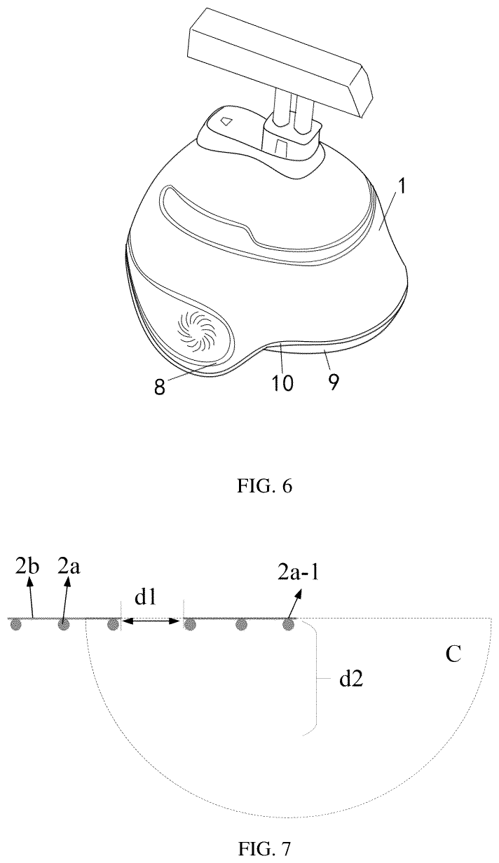

FIG. 6 shows a perspective view of a headgear of the phototherapy apparatus for treating Alzheimer's disease according to the embodiment of the present application. As shown in FIG. 6, the hood body 1 may have a left convex ear portion and a right convex ear portion 8, and the left convex ear portion is covered and is not shown in FIG. 6. The left convex ear portion and the right convex ear portion are designated collectively with the reference numeral 8. The left convex ear portion and right convex ear portion 8 may be constructed to extend down below the patient's ears to cover the patient's left temporal lobe and right temporal lobe, respectively. As shown in FIG. 1(b), when the phototherapy apparatus is standardly worn by patients, the distribution of near-infrared irradiation units 2 shown in A roughly corresponds to the area of the frontal lobe, and the distribution of near-infrared irradiation units 2 shown in B roughly corresponds to the area of the temporal lobe. The left convex ear portion and the right convex ear portion 8 are each equipped with near-infrared irradiation units 2 (as shown in FIG. 1(b)) to emit near-infrared light to the covered temporal lobe area. Referring to the distribution of brain regions shown in FIG. 2, the temporal lobe extends toward the ears, and this extension portion can be covered by the left and right convex ear portions 8 and be fully irradiated by the near-infrared light.

In some embodiments, the left convex ear portion and the right convex ear portion 8 can be constructed such that the near-infrared irradiation units 2 (as shown in FIG. 1(b)) arranged on the left convex ear portion and the right convex ear portion 8 can still irradiate the temporal lobe of the patient in the case that the head rotates within a predetermined angle range and moves in the up-down direction within a predetermined distance range during treatment. Specifically, the left convex ear portion and the right convex ear portion 8 can be designed to extend to the surrounding area of the head corresponding to the temporal lobe, keeping a margin associated with the predetermined angle range and predetermined distance range relative to the temporal lobe. In this way, even if the patient rotates or moves due to moving willingness or uncontrollable tremors, his/her temporal lobe can always be fully irradiated to ensure the treatment effect.

The left and right convex ear portions 8 can extend toward the patient's ears, and in some embodiments, can extend downwards below the patient's ears, so that the near-infrared light emitted by the near-infrared irradiation units 2 arranged on the left temporal lobe and the right temporal lobe can not only comprehensively irradiate the left and right temporal lobes, but also irradiate the hippocampus through the ear canal. Please note that the hippocampus can be reached from the ear to the deep along the ear canal, and the light transmission distance by way of the ear canal to the hippocampus is much smaller than the light transmission distance from the frontal lobe to the hippocampus, and the attenuation of the near-infrared light in the ear canal is much smaller than that in the skull. In this way, the hippocampus located deep in the brain can also be fully irradiated by the near-infrared light. The hippocampus is closely related to the development of AD. By enabling enough near-infrared light energy to reach the frontal lobe and temporal lobe meanwhile reaching the hippocampus, it can significantly improve the function of brain mitochondria and the level of ATP, promote the decomposition of β-amyloid protein (Aβ), reduce the deposition of Aβ, reduce the damage to nerve cells, improve the repair and regeneration ability of nerve tissue, and improve cognitive ability, such that the phototherapy effect on AD is more significant.

In some embodiments, the hood body 1 includes a forehead portion 9, and has a curved connecting portion 10 between the forehead portion 9 and the left convex ear portion and the right convex ear portion 8, so that the lower edges of the left convex ear portions 8, the forehead portion 9 and the right convex ear portion 8 are connected into a whole in a curved manner so as to completely cover the left temporal lobe and the right temporal lobe of the patient. As shown in the distribution of brain regions in FIG. 2, a part of the temporal lobe is near the temple, by means of the curved connecting portion 10, this part can be covered and sufficient near-infrared light irradiation can be provided. The inventors found that, with the development of AD, the lesions will spread to various parts of the temporal lobe, providing thoughtful and sufficient near-infrared light irradiation to each part can achieve a more effective treatment effect. Sometimes, it is impossible to determine the specific part of the temporal lobe involved in the lesion without cerebral functional imaging. For patients, the cost for acquiring cerebral functional imaging is high. In addition, the shape, size, etc., of the head of each patient may vary. And when the phototherapy apparatus is actually worn, since the phototherapy apparatus is worn on the head of the patient, it is difficult for medical staff, patients or other accompanying personnel to accurately determine the position of each brain region from the external surface of the head. Then, with the joint design of the left convex ear portion and the right convex ear portion 8 and the curved connecting portion 10, it can completely cover all parts involved by the lesion, so as to achieve a more effective treatment effect, and there is no need to distract the attention of the accompanying personnel or patients themselves, which can reduce the workload.

In some embodiments, the lower edge of the front portion of the hood body 1 is in a gentle curved shape, and the middle portion of the lower edge extends downward relative to the two side portions, so as to guide the user to wear the lower edge to the brow bone. This curved shape with a lower middle and two slightly higher sides matches the configuration of the brow bone. According to daily habits (such as the habit of wearing glasses), users will naturally pull the curved lower edge closer to the brow bone, so, the complete forehead can be irradiated, and the doctor or patient can visually confirm that the wearing position is appropriate. For the loosely designed hood body 1, this curved shape with a lower middle and two slightly higher sides will also guide the user's forehead to actively approach the hood body 1 when wearing it. This wearing position close enough to the forehead is appropriate, because the frontal lobe is the key treatment area, which needs to ensure the irradiation effect, and by bringing the lower edge of the curved shape close to the brow bone, both the patient and the doctor can confirm that the appropriate wearing position has been reached, and the patient will consciously maintain the appropriate wearing position.

Returning to FIG. 1(b), the irradiation parameters of at least part of the near-infrared irradiation units 2 are adjustable independently, and the irradiation parameters include at least the average irradiance. For example, the irradiation parameters of each near-infrared irradiation unit 2 can be independently controlled. That is, the irradiation parameters are adjusted in units of the near-infrared irradiation unit 2, and the near-infrared irradiation unit 2 may include a group of a plurality of near-infrared light emitting diodes 2a, so that the irradiation parameters of all the near-infrared light emitting diodes 2a can be controlled flexibly and efficiently. In addition to the average irradiance, the irradiation parameters may also include pulse frequency, waveform, duty cycle, etc. In some other embodiments, the irradiation parameters of every two near-infrared irradiation units 2 can also be controlled independently, which can be determined according to actual irradiation requirements. In some embodiments, the pulse frequency of at least part of the near-infrared irradiation units 2 is adjustable independently. In this way, by setting different pulse frequencies for different brain regions, targeted treatment may be performed for each brain region.

In some embodiments, the array of near-infrared irradiation unit 2 is constructed to emit near-infrared light with a central wavelength of 800 nm-820 nm to the patient's head. The study found that it is more suitable and effective to use single wavelength near-infrared light with a central wavelength of 800 nm-820 nm.

FIG. 3(a) shows a comparative diagram of energy deposition in the dorsal lateral prefrontal cortex (dlPFC) of individuals of different ages when they are irradiated with near-infrared light of various wavelengths according to the embodiment of the present application, and FIG. 3(b) shows a comparative diagram of energy deposition in the ventromedial prefrontal cortex (vmPFC) of individuals of different ages when they are irradiated with near-infrared light of various wavelengths according to the embodiment of the present application. As shown in FIG. 3(a) and FIG. 3(b), for the energy deposition in the two cortex regions of dlPFC and vmPFC in different age ranges, the energy deposition for the central wavelength of 810 nm is better than that of 670 nm, 850 nm, 980 nm and 1064 nm, and the next suboptimal are the central wavelengths of 1064 nm and 850 nm. Simulation experiments have verified that the energy deposition conditions of single wavelength near-infrared light with other wavelength values in the central wavelength of 800 -820 nm can also acquire better energy deposition conditions than 670 nm, 850 nm, 980 nm and 1064 nm.

The near-infrared irradiation unit 2 of the embodiment of the present application uses near-infrared light with a single wavelength and a central wavelength of 800-820 nm for irradiation, and its central wavelength is neither in the wavelength range around 670 nm (for example, 630-750 nm), nor the wavelength range around 980 nm (for example, 900-1020 nm), so that an optimized energy deposition condition can be acquired. Furthermore, the near-infrared light emitted by the near-infrared irradiation unit 2 of the embodiment of the present application does not depend on dual wavelengths or multi-wavelengths. A single wavelength with a central wavelength of about 810 nm can achieve good treatment effect as long as the irradiance is appropriate. Animal experiment data and clinical data will be provided to confirm this herein after.

FIG. 4 shows the absorption curves diagram of near-infrared light of different wavelengths in water, deoxyhemoglobin, and oxyhemoglobin according to the embodiment of the present application. As shown in FIG. 4, when the wavelength of 950 nm-1000 nm is used, the absorption rate of near-infrared light in water is very high, but the absorption rate in deoxyhemoglobin is very low, which is much lower than the absorption rate of near-infrared light with a wavelength of about 810 nm in deoxyhemoglobin. It can also be seen from FIG. 4 that the absorption rate of near-infrared light with the wavelength range of 800-820 nm in deoxyhemoglobin and oxyhemoglobin is relatively balanced and significantly higher than that in water. For the same treatment target region, if the near-infrared light with the same irradiance is used to irradiate, obviously, using dual wavelengths such as 760 nm-860 nm and 950-1000 nm is not as effective as focusing on using single wavelength around 810 nm for irradiation. The absorption effect of oxyhemoglobin and deoxyhemoglobin is better, and the treatment effect is also better. The cost of the array of single wavelength near-infrared irradiation unit 2 is lower and it is more convenient to control.

Near-infrared light may include pulsed light. A single-frequency pulsed light within an appropriate frequency range has a good irradiation effect, and the frequency range of the single frequency can be the frequency range of a wave (for example, 10 Hz) or the frequency range of y wave (for example, 40 Hz). In some embodiments, irradiation can be performed using pulsed light containing at least two frequency components within a certain frequency range, and in some cases, the irradiation effect is better than that of conventional single-frequency pulsed light. In some embodiments, the pulsed light includes a pulse wave component at a first pulse frequency and/or a pulse wave component at a second pulse frequency, the first pulse frequency is 7 Hz-13 Hz, and the second pulse frequency is 30 Hz-100 Hz. Preferably, the first pulse frequency is 10 Hz, and the second pulse frequency is 40 Hz. The present invention finds that using appropriate frequencies, such as but not limited to α-wave frequency band and γ-wave frequency band, has a better irradiation effect than other frequencies. The pulsed light includes α wave and γ wave as pulse wave component at the first pulse frequency and pulse wave component at the second pulse frequency, respectively, and is formed by any of the following methods. For example, the α wave and the γ wave can be synchronously mixed to form the waveform of the pulsed light emitted by each near-infrared light emitting diode 2a. That is to say, let each near-infrared light-emitting diode 2a directly emit the waveform formed by synchronous mixing of α wave and γ wave, so as to realize the time-synchronized and spatially overlapping mixed waveform (pulsed light mixed by two frequency bands of α wave and γ wave). For another example, the α wave and γ wave can be time-divisionally combined to form the waveform of the pulsed light emitted by each near-infrared light emitting diode 2a. That is to say, for the same near-infrared light-emitting diode 2a, it can be turned on at different time periods to alternately irradiate α wave and γ wave, which is a mixed mode with pure spatial overlap. In some embodiments, each of the first group of near-infrared light emitting diodes 2a can be controlled to emit pulsed light of α wave, and each of the second group of near-infrared light-emitting diodes 2a emits pulsed light of γ wave synchronously therewith. That is, let the first group and the second group of the near-infrared light-emitting diodes 2a be turned on simultaneously at the same time period. Specifically, the near-infrared light-emitting diodes 2a corresponding to different brain regions can be turned on at the same time period, which is a mixed mode with only temporal overlapping, so that different brain regions can be provided with pulsed light of specific frequency and waveform.

Please note that in the present application, a loose and open headgear is used as the carrier for irradiating multi-frequency pulsed light, but the present application is not limited thereto, and headgears of other structures can also be used, such as a headgear that fits the head, or a non-headgear wearing device, such as but not limited to glasses, wearing devices installed through the ear canal or nasal cavity, etc., are used as a carrier to irradiate multi-frequency pulsed light towards the patient's brain.

In some embodiments, the pulse frequency of the near-infrared irradiation unit 2 is adjustable, wherein the adjustable range is 0 Hz-100 Hz, for example, near-infrared light with a pulse frequency of 8 Hz, 10 Hz, 30 Hz, 40 Hz, etc is used to perform irradiation. In this way, when using phototherapy apparatus to implement phototherapy, the user can independently and specifically determine a more appropriate pulse frequency according to the degree of disease, the target brain region, etc., so as to achieve a better phototherapy effect.

The present application also provides a headgear of a phototherapy apparatus for treating Alzheimer's disease. As shown in FIGS. 1(a) and 1(b), the headgear includes a hood body 1 that loosely accommodates the patient's head, so as to enable the head to rotate within a predetermined angle range and move in the up-down direction within a predetermined distance range during the treatment. The headgear also includes an array of near-infrared irradiation units 2 arranged in the hood body 1, and the array of near-infrared irradiation units 2 is constructed to emit near-infrared light to the patient's head. In some embodiments, each near-infrared irradiation unit 2 may include a plurality of near-infrared light emitting diodes 2a. The implementation methods of the array of near-infrared irradiation units 2 described in various embodiments of this application can be combined herewith.

The headgear may also include a cooled gas delivering inner cavity 5 disposed adjacent to the array of near-infrared irradiation units 2 in the hood body 1 and a cooled gas delivering pathway 7 leading from the cooled gas delivering inner cavity 5 to the patient's head. The cooled gas generated by the refrigerator 3 outside the headgear is sent into the cooled gas delivering inner cavity 5 and blown towards the patient's head through the cooled gas delivering pathway 7 to dissipate heat from the patient's head.

Now returning to FIG. 1(b), the specific implementation methods of near-infrared irradiation unit 2 and cooling mechanism 4 in the headgear of a phototherapy device for treating Alzheimer's disease will be illustrated.

As shown in FIG. 1(b), the headgear may include a hood body 1 that loosely accommodates the patient's head so as to enable the head to rotate within a predetermined angle range and move in the up-down direction within a predetermined distance range during the treatment. The implementation of the hood body 1 described in connection with the phototherapy apparatus in each embodiment of the present application can be combined here, and will not be repeated here.

The headgear is also provided with a cooling component used in cooperation with the external refrigerator 3 and the cooled gas delivery pipeline 12 to form a cooling mechanism 4 capable of sufficiently dissipating heat from the patient's head. Specifically, as shown in FIG. 1(b), a cooled gas delivering inner cavity 5 and a cooled gas delivering pathway 7 leading to the head of the patient from the cooled gas delivering inner cavity 5 are provided near the array of near-infrared irradiation units 2 in the hood body 1, so that the cooled gas generated by the refrigerator 3 outside the headgear is sent into the cooled gas delivering inner cavity 5 (for example, via the cooled gas delivery pipeline 12) and blown to the patient's head through the cooled gas delivering pathway 7 to dissipate heat from the patient's head.

Combining FIG. 1(a) and FIG. 1(b), the hood body 1 includes an outer layer 12a and a cover with a certain light transmittance 13 as an inner layer, and a cooled gas delivering inner layer 5 is formed between the outer layer 12a and the cover with a certain light transmittance 13. The cover with a certain light transmittance 13 is provided with a plurality of ventilation holes 6, so that each ventilation hole 6 together with the gap between the cover with a certain light transmittance 13 and the patient's head forms the cooled gas delivering pathway 7. It can be seen that the ventilation holes 6 can be provided in groups, so that the cooled gas blown to the head from the surroundings can be distributed more evenly, so as to enable AD patients feel comfortable and cooperate with the treatment. The cover with a certain light transmittance 13 may be transparent or translucent.

The near-infrared irradiation unit 2 can be implemented in various ways, for example, it can be a lamp panel 2b carrying a plurality of near-infrared light-emitting diodes 2a (as shown in FIG. 1(b)), and the plurality of ventilation holes 6 are distributed in groups (as shown in FIG. 1(a)), which can be implemented as a multi-point array, so that each group of ventilation holes 6 corresponds to each lamp panel 2b, respectively. The gap part facing the lamp panel 2b is significantly heated, and each group of ventilation holes 6 corresponding to each lamp panel 2b can deliver cooled gas in a targeted manner to effectively reduce the heat in this part of the gap, and the cooled gas can be discharged smoothly and evenly through each ventilation hole 6 to evenly dissipate heat from the scalp, which can further improve the patient's comfort.

The outer layer 12a of the hood body 1 includes a hot gas extraction inner cavity 14, the hot gas extraction inner cavity 14 at least partially accommodates a circuit 15, and is interconnected to the outside via the air inlet 16 (as shown in FIG. 5) and the air outlet 17, so that the air introduced through the air inlet 16 carries the heat generated by the circuit 15 and is discharged to the outside through the air outlet 17. The inventor noticed that when the array of near-infrared irradiation units 2 is constructed so that the average irradiance is greater than 40 mW/cm², the heat production of the circuit 15 will also be very high, and sometimes the local heat production of the circuit 15 is significantly higher than the heat production of the photothermal conversion of the near-infrared light-emitting diode 2a. Thus, the hot gas extraction inner cavity 14 is respectively provided to efficiently remove the heat, so as to prevent the local heat production of the circuit 15 from being conducted to the near-infrared light-emitting diode 2a side or even the head side in large amount, and thus improving heat dissipating efficiency. In some embodiments, the outer side of the circuit 15 may be equipped with a heat conduction sheet to guide heat to be transferred and discharged to the outer side.

In some embodiments, the hot gas extraction inner cavity 14 and the cooled gas delivering inner cavity 5 are independent of each other. In this way, the heat generated by the circuit 15 can be prevented from spreading to the cooled gas delivering inner cavity 5, affecting the heat dissipation effect on the gap between the head and the cover with a certain light transmittance 13.

In some embodiments, the hood body 1 may further include a spacer 18 which is used to devide the chamber inside the hood body 1 into a first chamber and a second chamber, the first chamber is used to accommodate a near-infrared irradiation unit 2, the second chamber is at least partially used as a cooled gas delivering inner cavity 5, and the wall of the second chamber on its side facing the head has light transmittance.

As an example, as shown in FIG. 1(a) and FIG. 1(b), the spacer 18 is located on the outside of the cooled gas delivering inner cavity 5, and each lamp panel 2b is located on the outside of the spacer 18. The spacer 18 can completely separate the hot gas extraction inner cavity 14 from the cooled gas delivering inner cavity 5. Thus, the transmitted cooled gas can be prevented from entering the hot gas extraction inner cavity 14, so that the cooled gas can act on the gap between the head and the cover with a certain light transmittance 13 to a greater extent, improving the heat dissipation effect on the gap. In addition, setting the lamp panel 2b in the hot gas extraction inner cavity 14 can keep the lamp panel 2b away from the head to a certain extent, thereby reducing the influence of the heat generated by the lamp panel 2b on the temperature of the head and ensuring a certain degree of safety. In order to ensure that the near-infrared light emitted by the lamp panel 2b can directly irradiate the scalp, similar to the cover with a certain light transmittance 13, the above-mentioned spacer 18 is also made of materials with a certain light transmittance to reduce the loss of near-infrared light, so that the near-infrared light can be directed toward the scalp as much as possible.

Note that the construction and quantity of spacer 18 are not limited to this. The spacer 18 can also include a set of curved components for forming separate multiple first chambers, and the space between the first chambers as well as the space between the first chambers and the inner cavity of the hood body 1 can be used as the second chamber for delivering cooled gas, which will not be further described here.

In some embodiments, the hot gas extraction inner cavity 14 and the cooled gas delivering inner layer 5 can also be interconnected. For example, a pipeline can be used to achieve interconnection. Specifically, the inner diameter of the pipeline can be set relatively small, so that more cooled gas in the cooled gas delivering chamber 5 is used to dissipate heat for the patient's head, and a small amount of cooled gas can enter the hot gas extraction inner cavity 14 through the pipeline to cool the circuit 15.

In some optional embodiments, the top portion of the cover with a certain light transmittance 13 is arch-shaped, and the curvature of the arch is smaller than a predetermined curvature, so that the introduced cooled gas diffuses gently to the periphery under the function of the arch-shaped top portion of the cover with a certain light transmittance 13.

The curvature of the above-mentioned arch can be as small as possible, so that the introduced large amount of cooled gas can be blown directly to the top of the cover with a certain light transmittance 13, and the flow rate of the cooled gas can be reduced under the action of the top of the cover with a certain light transmittance 13, and the cooled gas can spread around the top of the cover with a certain light transmittance 13, allowing the cooled gas to be smoothly and evenly discharged from each ventilation hole 6, improving the patient's comfort.

In some embodiments, there is a first predetermined distance between adjacent near-infrared irradiation units 2 in the array of near-infrared irradiation units 2. As an example, as shown in FIG. 1(b), the first predetermined distance between adjacent near-infrared irradiation units 2 is d. The adjacent near-infrared irradiation units 2 in the array cooperate to emit near-infrared light towards the patient's head at a predetermined divergence angle, so that in the case the patient's head remains still in the hood body 1 or moves within a movable gap, the emitted near-infrared light covers the target brain region and the average irradiance is greater than 40 mW/cm², which can ensure enough light energy to enter the brain tissue of the target brain region and ensure a good treatment effect. For example, when a phototherapy apparatus is used to treat AD, the target brain region includes all brain regions of the head, that is, near-infrared light covers the entire head, without leaving out any brain region.

Wherein, the first predetermined distance and the predetermined divergence angle may be set manually, or may be system default values. In this embodiment, the movable gap, the divergence angle, and the first predetermined distance between adjacent near-infrared irradiation units 2 are cooperatively configured so that the emitted near-infrared light forms an overlap of infrared light at the scalp after passing through the movable gap. This can not only cover the target brain region tightly, but also ensure sufficient average irradiance through overlapping.

Cooperatively configuring the movable gap, the divergence angle and the first predetermined distance can improve the treatment effect on AD. In some embodiments, in terms of cooperative configuration, the first predetermined distance, divergence angle, and movable gap of different brain regions can be changed according to the curvature of the hood body 1, the demand for average irradiance of different brain regions, and other related factors. For example, the area of the hood body 1 corresponding to the frontal lobe and the temporal lobe has a small curvature. At this time, the first predetermined distance between the adjacent near-infrared irradiation units 2 in the area of the hood body 1 corresponding to the frontal lobe and the temporal lobe can be set to be smaller than the first predetermined distance between adjacent near-infrared irradiation units 2 in other areas of the hood body 1 (such as the parietal lobe). If the movable gap is too large, it is difficult to realize that the near-infrared light emitted by the adjacent near-infrared irradiation unit 2 passes through the movable gap to form an appropriate overlap of infrared light on the scalp. For example, even if overlapping, excessive divergence angle can lead to insufficient average irradiance. For another example, if it is desired to strengthen the treatment effect on the frontal lobe and temporal lobe, and to apply a higher average irradiance to the frontal lobe and temporal lobe, at this time, the movable gap between the hood body 1 corresponding to the frontal lobe and temporal lobe and the head can be set smaller than the movable gaps corresponding to other brain regions, or the shape of the hood body 1 can guide the patient to move the head close to the frontal lobe and temporal lobe. For another example, the first predetermined distance between adjacent near-infrared irradiation units 2 in the area of the hood body 1 corresponding to the frontal lobe and temporal lobe can be reduced to enhance the treatment effect on the frontal lobe and temporal lobe.

By cooperatively configuring the movable gap, the first predetermined distance and the predetermined divergence angle, when the AD patient's head moves to the maximum in the hood body 1 (for example, close to the hood body 1 in a certain direction), it still covers completely the target brain regions. At the same time, the average irradiance can be ensured to be greater than 40 mW/cm² through overlapping', thereby ensuring the treatment effect on AD.

In some embodiments, there is a second predetermined distance between the array of near-infrared irradiation units 2 in the hood body 1 and the head, so that the near-infrared light emitted by adjacent near-infrared irradiation units 2 at a predetermined divergence angle overlaps at least partially in the projection area of the head, so that the target brain regions can all be irradiated by the near-infrared light. Wherein, the hood body 1 does not adopt an adaptive design, but adopts a loose design in which the head can move freely in the accommodating space, and the gap between the array of near-infrared irradiation units 2 in the hood body 1 and the head can cause the scattering of near-infrared light.

The inventor found through research that when the distance between the array of near-infrared irradiation units 2 and the head is too small, on one hand, the head of the patient has less movable space, which is less comfortable for AD patients and is not conducive for AD patients to coordinate with treatment. On the other hand, if the distance between the array of near-infrared irradiation units 2 and the head is small, the near-infrared light cannot be scattered sufficiently, causing the overlapping parts of the projection area of the near-infrared light emitted by adjacent near-infrared irradiation units 2 on the head are less, so that some parts of the head cannot be irradiated by near-infrared light, thereby reducing the treatment effect; or the arrangement density of near-infrared irradiation units 2 is required to be too high, which will lead to more heat generation and affect the patient's comfort, and the service life of the near-infrared irradiation unit 2 will be greatly reduced, and the manufacturing cost and control difficulty will be increased. On the contrary, if the distance between the array of the near-infrared irradiation units 2 and the head is too large, it will cause excessive divergence of the infrared light, resulting in a poor distribution balance of the average irradiance, or the average irradiance after superimposition is still insufficient, and still cannot get better treatment effect. By controlling the second predetermined distance between the array of near-infrared irradiation units 2 and the head, all parts of the head can be irradiated with near-infrared light with sufficient average irradiance and relatively uniform distribution. The required divergence angle range of the near-infrared irradiation unit 2 conforms to the emission angle range of conventional near-infrared LEDs (for example, 100° to 135°), and the required arrangement density and spacing of the near-infrared irradiation unit 2 are also relatively appropriate, such that the manufacturing difficulty and costs are beneficially controlled. Preferably, by setting the second predetermined distance between the array of near-infrared irradiation units 2 and the head to 2.5-4 cm, the lateral movable gap in the treatment process is controlled to 1-2 cm. The inventor conducted a survey and analysis of patients' psychology through clinical experiments, confirming that this is a psychologically comfortable range for patients. It can not only allow the patient to have enough freedom of movement, but also not be too empty to cause patients to be uneasy and worried about the inaccurate set-up of the head.

In some embodiments, the near-infrared irradiation unit 2 can be realized as a lamp panel 2b carrying a plurality of near-infrared LEDs 2a (as shown in FIG. 1(b). The near-infrared LEDs 2a are used to set the lamp panel 2b, and the divergence angle of the near-infrared LEDs 2a and the distance between the lamp panels 2b can be fully utilized to realize the overlapping of near-infrared light emitted by adjacent near-infrared LEDs 2a. Optionally, the distance between two adjacent near-infrared LEDs 2a is 12-13 mm. Moreover, the near-infrared LED 2a has an appropriate divergence angle, which can be coordinated with the movable gap and the first predetermined distance to improve the treatment effect. In the treatment of AD, emitting near-infrared light to the frontal lobe, temporal lobe and hippocampus together can have a better treatment effect. For key regions such as the frontal lobe and temporal lobe, a higher average irradiance is required, for example, more than 70 mW/cm² or even 150 mW/cm².

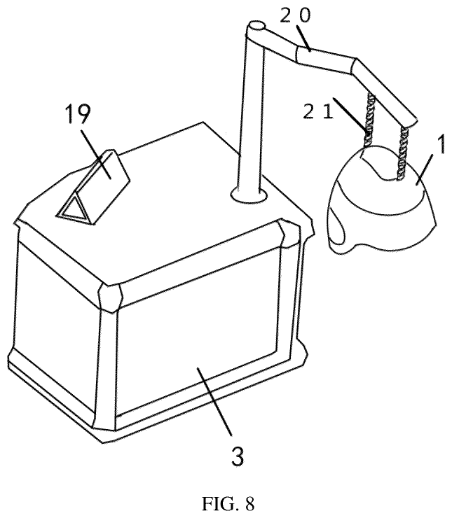

In some embodiments, in the corresponding regions of the frontal lobe and the temporal lobe, as shown in FIG. 7, there is a first predetermined spacing dl between two adjacent lamp panels 2b, such that the projection area on the head of the near-infrared light emitted by each near-infrared LED 2a (for example, LED 2a-1 in FIG. 7) on one of the lamp panels (e.g., the right lamp panel 2b in FIG. 7) on the head covers the projection area of the first predetermined spacing d1 on the head. Thus, the average irradiance of the encephalic regions corresponding to the frontal lobe and temporal lobe can be further increased, so that enough light energy can enter the brain region, thereby strengthening the treatment effect on the frontal lobe and temporal lobe, both of which are of interest.

For example, the first predetermined spacing can be set to be slightly smaller than the first predetermined distance between the lamp panels 2b arranged corresponding to other brain regions, so that the distribution density of near-infrared irradiation units 2 in the corresponding regions of the frontal lobe and temporal lobe is higher than that of the distribution density of corresponding regions in other brain regions. In addition, the first predetermined spacing may also be equal to the first predetermined distance. For example, even in the corresponding regions of the frontal lobe and the temporal lobe, since the corresponding regions of the frontal lobe and the temporal lobe have different curvatures, there may be the case that the first predetermined spacing is exactly equal to the first predetermined distance. Compared with setting the first predetermined distance, by means of further defining the first predetermined spacing between two adjacent near-infrared irradiation units 2 in the corresponding regions of the frontal lobe and the temporal lobe, it can further improve the average irradiance applied to the corresponding regions of the frontal lobe and temporal lobe and highlight the level of attention paid to the corresponding regions of the frontal lobe and temporal lobe.

In a specific embodiment, a plurality of LEDs 2a are arranged in the near-infrared irradiation unit 2, and there is a movable gap between the array of the near-infrared irradiation units 2 and the head. Therefore, the near-infrared light emitted by the LEDs 2a on each lamp panel 2b is scattered through the movable gap and projected to the head. In some embodiments, the first predetermined spacing d1 is 15-20 mm. Take the first predetermined distance of 15 mm as an example for illustration. In the case that the first predetermined spacing is 15 mm, the projection area of the near-infrared light emitted by the LED 2a farthest from the edge of the other lamp panel 2b in one of the adjacent lamp panels 2b on the head can also cover the projection area of the first predetermined spacing d1 on the head (as shown in area C in FIG. 7). At this time, it can ensure that the projection area of the near-infrared light emitted by each LED 2a on the head covers the projected area of the first predetermined spacing on the head, thereby improving the average irradiance in the corresponding areas of the frontal lobe and the temporal lobe and improving the treatment effect of AD.

Preferably, the first predetermined spacing d1 is 15-20 mm, the second predetermined distance d2 between each lamp panel 2b and the head is 2.5-4 cm, and the predetermined divergence angle is 100° to 135°, for example, 100°, 120°, 130°, etc., so that the deviation between the average irradiance of near-infrared light at the projection area of the first predetermined spacing on the head and the average irradiance at other positions of the head is less than 20%. Preferably, the deviation is less than 10%, in the case that it can be ensured that the emitted near-infrared light covers the target brain region and the average irradiance is greater than 40 mW/cm², so as to achieve focused irradiation on the corresponding regions of the frontal lobe and temporal lobe and ensure a higher and evenly distributed average irradiance in the corresponding regions of the frontal lobe and temporal lobe. Specifically, for example, when the average irradiance of the near-infrared light at the projection area of the first predetermined spacing on the head deviates by more than 20% from the average irradiance at other positions of the head, it is possible that the average irradiance of the near-infrared light at the projected area of the first predetermined spacing on the head is higher or lower, and correspondingly, the average irradiance at other positions is relatively lower or higher, resulting in uneven distribution of the average irradiance. And it will lead to overheating on the scalp where the average irradiance is higher (thus the treatment may have to be stopped), and insufficient treatment effect on the scalp where the average irradiance is lower, thereby reducing the overall treatment effect on the AD patient. In a preferred embodiment of the present application, a cooperative configuration is adopted as follows: the first predetermined spacing is set as 15-20 mm, the second predetermined distance between each lamp panel 2b and the head is set as 2.5-4 cm, and the predetermined divergence angle is set as 100° to 135°, for example, the divergence angle can be 120°. By considering the interaction among the first predetermined spacing, the second predetermined distance between each lamp panel and the head, and the predetermined divergence angle, it can ensure that the whole head is uniformly covered by near-infrared light and has a higher average irradiance.

FIG. 8 shows a overall structural schematic diagram of the phototherapy apparatus for treating Alzheimer's disease according to the embodiment of the present application. As shown in FIG. 8, the phototherapy apparatus may further include a user terminal 19, and the user terminal 19 may be configured to be interactively operated by a user. A computer storage medium may be configured in the user terminal 19, on which computer-executable instructions are stored, and when the computer-executable instructions are executed by a processor, various interaction steps with the user may be realized. The storage media may include read-only memory (ROM), flash memory, random-access memory (RAM), dynamic random-access memory (DRAM) such as synchronous DRAM (SDRAM) or Rambus DRAM, static memory (e.g., flash memory, static random-access memory), etc., on which computer-executable instructions may be stored in any format.

In some embodiments, the user terminal 19 can be configured to: acquire the physiological parameters of the patient, and the physiological parameters include age, light transmittance of extracellular tissue, and biochemical parameters related to Alzheimer's disease; generate a recommended infrared light treatment plan for the patient based on the obtained physiological parameters of the patient and display it to the user.

In some embodiments, the user terminal 19 is further configured to receive the user's confirmation operation on the proposed infrared light treatment scheme; after receiving the confirmation operation, each near-infrared irradiation unit 2 (as shown in FIG. 1(a) and FIG. 1(b)) performs irradiation according to the confirmed infrared light treatment scheme.

Specifically, the controller (not shown) for controlling the irradiation can be located on the user terminal 19, and can also be located at the headgear, and the user terminal 19 sends a execution instruction for confirming infrared light treatment scheme to the controller. The controller may be implemented by various processors, and may be a processing device including one or more general-purpose processing devices, such as a microprocessor, a central processing unit (CPU), a graphics processing unit (GPU), and the like. More specifically, the processor may be a Complex Instruction Set Computing (CISC) microprocessor, a Reduced Instruction Set Computing (RISC) microprocessor, a Very Long Instruction Word (VLIW) microprocessor, a processor running other instruction sets, or a processor that runs an instruction set combination. The processor can also be one or more special-purpose processing devices, such as an application-specific integrated circuit (ASIC), a field-programmable gate array (FPGA), a digital signal processor (DSP), a system on a chip (SoC), and the like. Preferably, most of the computing and processing are concentrated at the user terminal 19, so as to reduce the computing load and hardware and software costs of the headgear.

In some embodiments, the phototherapy apparatus further includes a bracket 20, as shown in FIG. 8, the hood body 1 is connected to the bracket 20 through an elastic member 21, and the elastic member 21 provides a certain amount of activity margin for the patient's head during treatment, so that the patient's head can move more freely, which can further improve the comfort and improve the patient's usage experience.

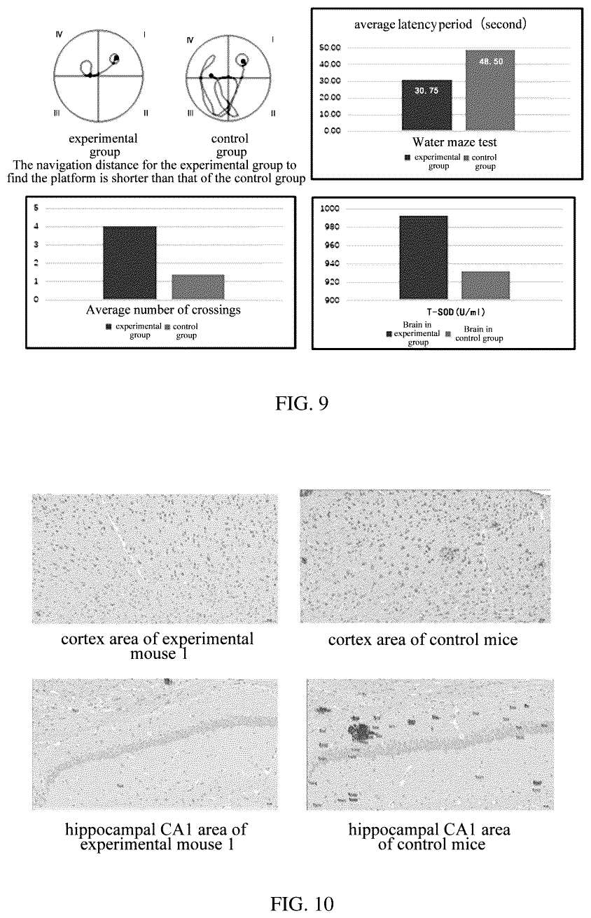

The inventor conducted phototherapy in vivo experiment on 5-month-old AD mice. The central wavelength of the used single wavelength near-infrared light was 800-820 nm, the pulse frequency was 10 Hz, and the frequency of irradiation was once a day. It lasted for 10 minutes every time and lasted for 5 weeks. The performance of the AD mice receiving phototherapy (also called the experimental group) was significantly improved in the water maze test compared to the AD mice in the control group (also called the control group). As shown in FIG. 9, compared to the control group, the experimental group significantly shortened the distance traveled to find the platform, and the experimental group shortened the escape latency by 36%. Moreover, the distribution of Aβ protein in the brain tissue of the experimental group (including both the cortical area and the hippocampal CA1 area) analyzed after sampling was significantly less than that of the control group, as shown in FIG. 10. Studies suggest that the extracellular accumulation of amyloid β (Aβ) protein produced by the cleavage of amyloid precursor protein (APP) is an important factor in the pathogenesis of AD. Extensive Aβ plaque deposition in the cerebral cortex and neurofibrillary tangles caused by tau protein lesions are significant pathological markers of AD. Therefore, the significantly reduced Aβ plaque deposition in the experimental group after the above-mentioned near-infrared light irradiation treatment means a good treatment effect on AD.

The inventor used the phototherapy apparatus according to the embodiment of the present application to conduct a clinical experiment of phototherapy for AD patients, used single wavelength near-infrared light with a central wavelength of 810 nm and an average irradiance greater than 40 mW/cm², and the irradiation frequency was 5-7 times a week, it lasted for 30 minutes every time, lasted for 4 months. Wherein, preferably, the average irradiance in key regions (the frontal lobe and temporal lobe) is about 90 mW/cm²-120 mW/cm². Wherein, the average optical irradiance in most regions is above 100 mW/cm². Preferably, the average irradiance in the parietal lobe and the occipital lobe was greater than 60 mW/cm².

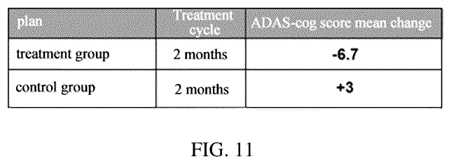

The results of the mid-term clinical trial are shown in FIG. 11, which shows a comparison chart of the Alzheimer's Disease Assessment Scale Cognitive Scale (ADAS-cog) scores between the treatment group of AD patients receiving phototherapy and control group of AD patients after performing phototherapy on AD patients using the phototherapy apparatus according to the embodiment of the present application.

It can be seen that after the treatment group received near-infrared phototherapy for 2 months, the total score of the ADAS-cog scale is decreased by 6.7 points on average compared with the baseline, with a significant statistical difference, while the total score of the ADAS-cog scale of the control group who did not receive near-infrared phototherapy is increased by 3 points after 2 months, and the results of the ADAS-cog score verified that the phototherapy apparatus according to the embodiment of the present application has a good treatment effect on AD.

In addition to the ADAS-cog score, this clinical trial also analyzed the MMSE score. The total score of the MMSE score is 30, and the lower the score is, the more severe the cognitive impairment is. The baseline MMSE score of AD patients who received phototherapy was 11.67 points, and the MMSE score after 2 months was 14.83 points, with an average increase of 3.12 points compared with the baseline, which indicates that after phototherapy, the cognitive function of AD patients is improved. The good treatment effect of the phototherapy apparatus according to the embodiment of the present application on AD was also verified by the MMSE score.

In some embodiments, the phototherapy apparatus for treating Alzheimer's disease according to the present application can be used for treating mental diseases and mental disorders such as depression, autism, and bipolar disorder.

The inventors have confirmed through clinical experiments that using higher average irradiance to treat mental diseases and mental disorder such as depression can achieve better treatment effect while ensuring safety and patient's comfort. In the treatment of depression, it is preferable that the average irradiance of near-infrared light emitted at least towards the frontal lobe of the head is 80 mW/cm²-250 mW/cm². In another embodiment, near-infrared light with an average irradiance of 80 mW/cm²-250 mW/cm² can also be emitted simultaneously to the frontal lobe and the temporal lobe to achieve better phototherapy effect on depression.

In some embodiments, in the case of treating depression, the array of near-infrared irradiation units 2 is constructed to emit near-infrared light to at least some nodes of the brain networks, the brain networks at least include at least one of the default mode network, salience network and central executive network. It can be understood that the brain network may include a plurality of nodes with brain regions as nodes, and various nodes corresponds to different brain regions, or a plurality of nodes may be set on one brain region, and there is a functional connection between the node pairs. The functional connection strength between node pairs can be used to characterize the collaborative work and information transmission between brain regions.

For autistic patients, in all age groups of the autistic patients, the frontal lobe, temporal lobe, hippocampus, amygdala, corpus callosum, etc., often have different degrees of structural, functional and connection abnormalities, and similar to the treatment of depression, the treatment of autism also needs a high average irradiance, and even a higher average irradiance is required to irradiate brain regions associated with autism for better treatment effect. Therefore, in the case of treating autism, the phototherapy apparatus of the present application can make the average irradiance of the near-infrared light emitted by the array of the near-infrared irradiation units 2 corresponding to the brain region associated with autism be 100 mW/cm²-250 mW/cm². When using phototherapy apparatus to perform phototherapy on such autistic patients, the average irradiance of near-infrared light emitted by the array of the near-infrared irradiation units 2 corresponding to one or more brain regions in the frontal lobe, temporal lobe, hippocampus, amygdala and corpus callosum can be 100 mW/cm²-250 mW/cm². In some embodiments, when using phototherapy apparatus to treat autism, we can also carry out targeted therapy for local targets of autism based on the brain network related to autism.

In the case of treating bipolar disorder, the average irradiance of near-infrared light emitted by the array of near-infrared irradiation units 2 corresponding to the brain region associated with bipolar disorder is 100 mW/cm²-250 mW/cm², the brain regions associated with bipolar disorder may include the frontal lobe and limbic brain regions, such as the ventrolateral prefrontal cortex, dorsal lateral prefrontal cortex, intraparietal sulcus, and the like. It is understandable that, unlike other mental diseases and mental disorders, bipolar disorder has different periods, including a depressive period and a manic period, and the abnormality of brain networks will also vary when patients are in different periods. For example, targeted light irradiation therapy for abnormalities of brain networks at different periods can achieve more precise treatment of bipolar disorder.

It can be understood that when using brain networks to implement precise phototherapy for mental diseases and mental disorders such as depression, autism, bipolar disorder, etc., the irradiation parameters of near-infrared light can be specifically set based on the type of disease, degree of illness, functional connectivity between nodes of the brain network, etc. This application does not make specific restrictions on this. wherein, the irradiation parameters can include central wavelength, average irradiance, pulse frequency, etc.

In some embodiments, the above structures of the left convex ear portion and the right convex ear portion 8 can be used not only for treating AD, but also for treating other diseases related to the frontal and temporal lobes, such as depression.

Furthermore, although exemplary embodiments have been described herein, their scope includes any and all embodiments having equivalent elements, modifications, omissions, combinations (e. g., schemes crossed by various embodiments), adaptations or changes based on the present application. The elements in the claims will be interpreted broadly based on the language used in the claims, not limited to the examples described in the description or during the implementation of this application, and the examples will be interpreted as non-exclusive. Therefore, the specification and examples are intended to be considered as examples only, and the true scope and spirit are indicated by the following claims and the full scope of their equivalents.

The above description is intended to be illustrative rather than restrictive. For example, the above examples (or one or more of them) may be used in combination with each other. For example, those skilled in the art may use other embodiments when reading the above description. In addition, in the above specific embodiment, various features can be grouped together to simplify this application. This should not be interpreted as the intention that a public feature that does not require protection is necessary for any claim. On the contrary, the subject matter of the present application may be less than all the features of a particular disclosed embodiment. Thus, the following claims are hereby incorporated into the specific embodiments as examples or embodiments, wherein each claim is independently regarded as a separate embodiment, and it is considered that these embodiments can be combined with each other in various combinations or arrangements. The scope of the present invention shall be determined by reference to the full scope of the appended claims and the equivalent forms empowered by these claims.

The above embodiments are only exemplary embodiments of this application and are not intended to limit this application. The protection scope of the invention is defined by the claims. Those skilled in the art may make various modifications or equivalent substitutions to this application within the substance and protection scope of this application, and such modifications or equivalent substitutions shall also be deemed to fall within the protection scope of the application.

1. A phototherapy apparatus for treating Alzheimer's disease, comprising:
a hood body that loosely accommodates, rather than adapts to, a patient's head, so as to enable the head to rotate within a predetermined angle range and move in an up-down direction within a predetermined distance range during treatment;
an array of near-infrared irradiation units arranged in the hood body, a light transmittable portion is provided at the inner side of the array of near-infrared irradiation units, which is constructed to emit via the light transmittable portion near-infrared light with an emergent average irradiance greater than 40 mW/cm² towards the patient's head, and a total power of which is 10 W or more; and
a cooling mechanism configured to introduce cooled gas and send it into an accommodation cavity of the hood body for accommodating the patient's head, blowing it towards the patient's head through a cooled gas delivering pathway between the hood body and the patient's head to dissipate heat from the patient's head.

2. The phototherapy apparatus according to claim 1, wherein the cooling mechanism comprises a refrigerator or is connectable to a refrigerator, the hood body is provided with a cooled gas delivering inner cavity to receive cooled gas generated by the refrigerator, and the cooled gas delivering pathway is formed between the cooled gas delivering inner cavity and the patient's head.

3. The phototherapy apparatus according to claim 1, wherein the array of near-infrared irradiation units is constructed to emit near-infrared light with a central wavelength of 800-820 nm towards the patient's head; and/or the array of near-infrared irradiation units is constructed to emit single wavelength near-infrared light with a central wavelength of 810 nm towards the patient's head.

4. (canceled)

5. The phototherapy apparatus according to claim 1, wherein the near-infrared light is a pulsed light, and irradiation parameters of a near-infrared irradiation unit of the array of near-infrared irradiation units further include a pulse frequency, the pulsed light includes a pulse wave component at a first pulse frequency and/or a pulse wave component at a second pulse frequency, wherein the first pulse frequency is 7 Hz-13 Hz and the second pulse frequency is 30 Hz-100 Hz.

6. (canceled)

7. The phototherapy apparatus according to claim 5, wherein each near-infrared irradiation unit of the array of near-infrared irradiation units comprises a plurality of near-infrared light-emitting diodes, and the pulsed light comprises pulse wave components with a wave frequency and y wave frequency as the first pulse frequency and the second pulse frequency, respectively, and is formed by any of the following manners:
a waveform of the pulsed light emitted by each near-infrared light-emitting diode is formed by synchronous aliasing of α wave and γ wave;
the waveform of the pulsed light emitted by each near-infrared light-emitting diode is formed by time division combination of α wave and γ wave;
each near-infrared light-emitting diode in a first group of the plurality of near-infrared light-emitting diodes emits pulsed light of α wave, each near-infrared light-emitting diode in a second group of the plurality of near-infrared light-emitting diodes emits pulsed light of γ wave, and the first and second groups of near-infrared light-emitting diodes emit pulsed light synchronously.

8. The phototherapy apparatus according to claim 1, wherein the cooling mechanism is further configured to blow the cooled gas towards the patient's head through the cooled gas delivering pathway with a wind speed of 0.5 m/s to 3.5 m/s; and/or
maintain a surface temperature of the light transmittable portion near a scalp of the patient not higher than 43° C. under a condition that the phototherapy apparatus irradiates continuously for 30 minutes.

9. (canceled)

10. The phototherapy apparatus according to claim 1, wherein the phototherapy apparatus is configured to set nodes of a brain network for targeted irradiation and irradiation parameters, based on functional connection parameters of the brain network of the patient related to Alzheimer's disease.

11. The phototherapy apparatus according to claim 1, wherein the hood body is constructed to cover at least a whole brain of the patient, without covering eyes of the patient.

12. The phototherapy apparatus according to claim 10, wherein the phototherapy apparatus is further configured to: under the condition that a node of the brain network for targeted irradiation is located deeply inside the brain, irradiate another node, which is easier to irradiate and forms a functional connection with the node located deeply.

13. The phototherapy apparatus according to claim 1, wherein the emergent average irradiance of the near-infrared light emitted to a frontal lobe via the light transmittable portion is in the range of 50 mW/cm² to 250 mW/cm²; and/or
the emergent average irradiance of the near-infrared light emitted to a temporal lobe via the light transmittable portion is in the range of 70 mW/cm² to 250 mW/cm².

14. The phototherapy apparatus according to claim 1, wherein the phototherapy apparatus is further configured to: based on other psychological disorder of the patient, regulate an irradiation parameter of the near-infrared light irradiated to the patient's brain network nodes related to an other psychological disorder, including an average irradiance; or
under the condition that the patient has other psychological disorder, cause the array of the near-infrared irradiation units to emit via the light transmittable portion the near-infrared light to the patient's brain network nodes related to the other psychological disorder, with the emergent average irradiance in the range of 80 mW/cm² to 250 mW/cm².

15. The phototherapy apparatus according to claim 1, wherein the array of near-infrared irradiation units is constructed to emit near-infrared light to nodes of a brain network related to development of Alzheimer's disease;
or emit near-infrared light to the nodes of the brain network which include at least one of medial prefrontal cortex, medial temporal lobe, cingulate cortex, precuneus, and inferior parietal lobe.

16. The phototherapy apparatus according to claim 15, wherein the phototherapy apparatus is further configured to: cause the near-infrared irradiation units to emit near-infrared light to the nodes of the brain network, the functional connection strength of which with a hippocampus is weaker than a predetermined level, and a functional connection strength of each node with the hippocampus is determined by cerebral functional imaging of the patient.

17. The phototherapy apparatus according to claim 1, wherein the hood body has a left convex ear portion and a right convex ear portion, and the left convex ear portion and the right convex ear portion are constructed to extend downwards below a patient's ears, so as to cover a left temporal lobe and a right temporal lobe of the patient, respectively, the left convex ear portion and the right convex ear portion are each provided with near-infrared irradiation units to emit near-infrared light to the covered temporal lobe region.

18. The phototherapy apparatus according to claim 17, wherein the hood body further comprises a forehead portion, and has a curved connecting portion between the forehead portion and the left convex ear portion and the right convex ear portion, so that lower edges of the left convex ear portion, the forehead portion, and the right convex ear portion are connected into a whole in a curved manner, so as to completely cover the left temporal lobe and the right temporal lobe of the patient.

19. The phototherapy apparatus according to claim 2, wherein the hood body further includes a spacer, which is used to divide a chamber inside the hood body into a first chamber and a second chamber, wherein the first chamber is used to accommodate the array of near-infrared irradiation units, the second chamber is at least partially used as a cooled gas delivering inner cavity, and a wall of the second chamber on its side facing the patient's head has light transmittance.

20. The phototherapy apparatus according to claim 1, wherein the hood body comprises an outer layer and an inner layer, the light transmittable portion serves as the inner layer and forms an integrated light transmittable cover, a cooled gas delivering inner cavity is formed between the outer layer and the light transmittable cover, the light transmittable cover is provided with a plurality of ventilation holes, so that each ventilation hole and a gap, at level of multiple centimeters, between the light transmittable cover and the patient's head form the cooled gas delivering pathway.

21. (canceled)

22. The phototherapy apparatus according to claim 20, wherein the top portion of the light transmittable cover is arch-shaped with a curvature less than a predetermined curvature, so that the introduced cooled gas diffuses gently to a periphery under a function of the arch-shaped top portion.

23. The phototherapy apparatus according to claim 1, wherein there is a first predetermined distance between adjacent near-infrared irradiation units in the array of near-infrared irradiation units, there is a second predetermined distance between the array of near-infrared irradiation units and the head, and the adjacent near-infrared irradiation units cooperatively emit near-infrared light to the patient's head at a predetermined divergence angle, so that the emitted near-infrared light covers a target brain region under the condition that the patient's head remains stationary or moves within a movable gap within the hood body, and the near-infrared light emitted by adjacent near-infrared irradiation units at a predetermined divergence angle overlaps at least partially in a projection area of the head.

24. (canceled)

25. The phototherapy apparatus according to claim 23, wherein the near-infrared irradiation unit is a lamp panel carrying a plurality of near-infrared LEDs, and there is a first predetermined spacing between two adjacent near-infrared irradiation units in corresponding regions of a frontal lobe and temporal lobe, such that the projection area of the near-infrared light emitted by each near-infrared LED of one near-infrared irradiation unit on the patient's head covers the projected area of the first predetermined spacing on the patient's head.

26. (canceled)
